(19) United States
(12) Patent Application Publication
(10) Pub. No.: US 2009/0098078 A1
(43) Pub. Date: Apr. 16, 2009

(54) Water-In-Oil Microemulsions for Hair Treatment

(76) Inventors: Kelvin Brian Dickinson, Wirral (GB); Anand Ramchandra Mahadeshwar, Hamburg (DE); Ruby Loo Bick Tan-Walker, Wirral (GB)

Correspondence Address: UNILEVER PATENT GROUP, 800 SYLVAN AVENUE, AG West S. Wing, ENGLEWOOD CLIFFS, NJ, 07632-3100, US

(21) Appl. No.: 11/794,020

(22) PCT Filed: Nov. 24, 2005
(86) PCT No.: PCT/EP05/12849

§ 371 Date: Jan. 17, 2008

(30) Foreign Application Priority Data
Dec. 23, 2004 (EP) 04258087.8

(51) Int. Cl. A61K 8/06; A61Q 5/12

(52) U.S. Cl. 424/70.11; 424/70.1; 424/70.28

(57) ABSTRACT

The present invention provides a water-in-oil microemulsion for hair treatment comprising: (a) an oil phase comprising: (i) a first oily component which is one or more glyceride fatty esters, and (ii) a second oily component which is one or more hydrocarbon oils of average carbon chain length less than 20 carbon atoms, and (b) a hydrophilic phase comprising: (i) water, (ii) a nonionic emulsifier which is an ethoxylated alcohol having an HLB of at least 6, and (iii) a hair conditioning agent.

### FIELD OF THE INVENTION

This invention relates to water-in-oil microemulsions for hair treatment which have enhanced sensory properties and enhanced compatibility with hair benefit agents.

### BACKGROUND OF INVENTION AND PRIOR ART

Consumers oil hair both pre wash and post wash. Pre wash oiling is done as it is believed that oils nourish hair and protect it during the wash process. Post wash oiling is done for manageability and styling. The oiling habit is widely practised by around 800 million people across the Central Asia and Middle East region.

Coconut oil is by far the most common oil used in the Central Asia and Middle East region for hair care. It offers a high level of conditioning benefits, but with the drawback of greasy feel.

EP 1289479 discloses hair oils which incorporate a specific blend of oil types (glyceride fatty esters and hydrocarbon oils) and which can deliver an equivalent level of conditioning benefits to coconut oil, but with superior sensory properties, in particular less greasy feel.

It would be desirable to incorporate hair benefit agents such as hair conditioning agents into such oils, in order to improve the shine, feel and manageability of the hair after application of the product.

However a problem is that such agents are generally not compatible with the oil and cannot be incorporated into the oil in a stable manner. When such agents are combined with hair oils at effective levels, they tend to form a two-phase system, with an unattractive appearance and a tendency to separate due to differing density of the two phases.

The present inventors have found that this problem can be solved if a particular type of nonionic emulsifier is formulated with the oil. The invention provides an oil microstructure which has enhanced sensory properties and enhanced compatibility with hair benefit agents such as hair conditioning agents.

### DEFINITION OF THE INVENTION

The present invention provides a water-in-oil microemulsion for hair treatment comprising:

(a) an oil phase comprising:
(i) a first oily component which is one or more glyceride fatty esters, and
(ii) a second oily component which is one or more hydrocarbon oils of average carbon chain length less than 20 carbon atoms, and

(b) a hydrophilic phase comprising:
(i) water,
(ii) a nonionic emulsifier which is an ethoxylated alcohol having an HLB of at least 6, and
(iii) a hair conditioning agent.

### DETAILED DESCRIPTION OF THE INVENTION

Microemulsion

By “microemulsion” is meant a thermodynamically or kinetically stable liquid dispersion of an oil phase and a hydrophilic phase. The dispersed phase typically comprises small particles or droplets, with a size range of 5 nm to 200 nm, giving rise to a microemulsion that is transparent or translucent in appearance. This is in contrast to regular (macro-) emulsions that are turbid. The droplets or particles of the microemulsion may be spherical, although other structures are possible. The microemulsion is formed readily and sometimes spontaneously, generally without high-energy input.

(a) (i) Glyceride Fatty Ester

The water-in-oil microemulsion of the invention comprises an oil phase comprising a first oily component which is one or more glyceride fatty esters.

By “glyceride fatty esters” is meant the mono-, di-, and tri-esters formed between glycerol and long chain carboxylic acids such as C₆-C₃₀ carboxylic acids. The carboxylic acids may be saturated or unsaturated or contain hydrophilic groups such as hydroxyl.

Preferred glyceride fatty esters are derived from carboxylic acids of carbon chain length ranging from C₆ to C₂₄, preferably C₁₀ to C₂₂, most preferably C₁₂ to C₁₈.

Suitable glyceride fatty esters for use in microemulsions of the invention will generally have a viscosity at ambient temperature (25 to 30° C.) of from 0.01 to 0.8 Pa·s preferably from 0.015 to 0.6 Pa·s, more preferably from 0.02 to 0.065 Pa·s as measured by a Carri-Med CSL2 100 controlled stress rheometer, from TA Instruments Inc., New Castle, Del. (USA).

A variety of these types of materials are present in vegetable and animal fats and oils, such as camellia oil, coconut oil, castor oil, safflower oil, sunflower oil, peanut oil, cottonseed oil, corn oil, olive oil, cod liver oil, almond oil, avocado oil, palm oil, sesame oil, lanolin and soybean oil. These have various ranges of carbon chain lengths depending on the source, typically between about 12 to about 18 carbon atoms. Synthetic oils include trimyristin, triolein and tristearin glyceryl dilaurate. Vegetable derived glyceride fatty esters are particularly preferred, and specific examples of preferred materials for inclusion in microemulsions of the invention as sources of glyceride fatty esters include almond oil, castor oil, coconut oil, sesame oil, sunflower oil and soybean oil. Coconut oil, sunflower oil, almond oil and mixtures thereof are particularly preferred.

The glyceride fatty ester may be present in microemulsions of the invention as a single material or as a blend.

The total content of glyceride fatty ester in microemulsions of the invention suitably ranges from 10% to 95%, preferably from 20% to 80%, by weight based on total weight of the microemulsion.

(a)(ii) Hydrocarbon Oil

The oil phase of the water-in-oil microemulsion of the invention comprises a second oily component which is one or more hydrocarbon oils of average carbon chain length less than 20 carbon atoms.

Suitable hydrocarbon oils include cyclic hydrocarbons, straight chain aliphatic hydrocarbons (saturated or unsaturated), and branched chain aliphatic hydrocarbons (saturated or unsaturated). Straight chain hydrocarbon oils will typically contain from about 6 to about 16 carbon atoms, preferably from about 8 up to about 14 carbon atoms. Branched chain hydrocarbon oils can and typically may contain higher numbers of carbon atoms, e.g. from about 6 up to about 20 carbon atoms, preferably from about 8 up to about 18 carbon atoms.

Suitable hydrocarbon oils will generally have a viscosity at ambient temperature (25 to 30° C.) of from 0.0001 to 0.5 Pa·s, preferably from 0.001 to 0.05 Pa·s, more preferably from 0.001 to 0.02 Pa·s as measured by a Carri-Med CSL2 100 controlled stress rheometer, from TA Instruments Inc., New Castle, Del. (USA).

A preferred hydrocarbon oil is light mineral oil. Mineral oils are clear oily liquids obtained from petroleum oil, from which waxes have been removed, and the more volatile fractions removed by distillation. The fraction distilling between 250° C. to 300° C. is termed mineral oil, and it consists of a mixture of hydrocarbons, in which the number of carbon atoms per hydrocarbon molecule generally ranges from C₁₀ to C₄₀. Mineral oil may be characterised in terms of its viscosity, where light mineral oil is relatively less viscous than heavy mineral oil, and these terms are defined more specifically in the U.S. Pharmacopoeia, 22nd revision, p. 899 (1990). A commercially available example of a suitable light mineral oil for use in the invention is Sirius M40 (carbon chain length C₁₀-C₂₈, mainly C₁₂-C₂₀, viscosity 4.3×10⁻³ Pa·s), available from Silkolene.

Other hydrocarbon oils that may be used in the invention include relatively lower molecular weight hydrocarbons including linear saturated hydrocarbons such a tetradecane, hexadecane, and octadecane, cyclic hydrocarbons such as dioctylcyclohexane (e.g. CETIOL S from Henkel), branched chain hydrocarbons (e.g. ISOPAR L and ISOPAR V from Exxon Corp.).

The hydrocarbon oil may be present in microemulsions of the invention as a single material or as a blend.

The total content of hydrocarbon oil in microemulsions of the invention suitably ranges from 5% to 90%, preferably from 20% to 80%, by weight based on total weight of the microemulsion.

The glyceride fatty ester:hydrocarbon oil weight ratio in microemulsions of the invention may suitably range from 90:10 to 10:90, preferably from 80:20 to 20:80, more preferably from 60:40 to 40:60. Particularly preferred are blends of [coconut oil and/or sunflower oil and/or almond oil] and light mineral oil, in which the [coconut oil and/or sunflower oil and/or almond oil]:light mineral oil weight ratio is about 50:50.

(b)(i) Water

The hydrophilic phase of the water-in-oil microemulsion of the invention comprises water, suitably at a level of from about 2% by weight based on total weight of the microemulsion. Suitably the water level does not exceed about 10% by weight based on total weight of the microemulsion, since this may lead to a hazy product appearance which is undesirable to consumers of hair oils. Preferably the water level ranges from 3 to 7%, more preferably from 4 to 6% by weight based on total weight of the microemulsion.

(b)(ii) Nonionic Emulsifier

The water-in-oil microemulsion of the invention comprises a nonionic emulsifier which is an ethoxylated alcohol having an HLB of at least 6.

Suitable ethoxylated alcohols are commercially available and include the primary aliphatic alcohol ethoxylates and secondary aliphatic alcohol ethoxylates. The length of the polyethenoxy chain can be adjusted to achieve the desired balance between the hydrophobic and hydrophilic elements.

The HLB value of the ethoxylated alcohol suitably ranges from 6 to 12, preferably from 7 to 10, more preferably from 7 to 9.

Examples of suitable ethoxylated alcohols include the condensation products of a higher alcohol (e.g., an alkanol containing about 8 to 16 carbon atoms in a straight or branched chain configuration) condensed with about 2.5 to 20 moles of ethylene oxide.

A preferred group of the foregoing ethoxylated alcohols are the Neodol ethoxylates (Shell Co.), which are higher aliphatic, primary alcohols containing about 9 to 15 carbon atoms condensed with about 2.5 to 20 moles of ethylene oxide. Specific examples are C9 to 11 alkanol condensed with 2.5 to 10 moles of ethylene oxide (Neodol 91-8 or Neodol 91-5), C12 to 13 alkanol condensed with 3 moles ethylene oxide (Neodol 23-3), C12 to 15 alkanol condensed with 12 moles ethylene oxide (Neodol 25-12), C14 to 15 alkanol condensed with 13 moles ethylene oxide (Neodol 45-13), and the like.

Such ethoxylates have an HLB (hydrophobic lipophilic balance) value of about 7 to 10. Most preferred is Neodol 23-3, with an HLB of about 8.

The level of nonionic emulsifier in microemulsions of the invention suitably ranges from 10 to 40%, preferably from 15 to 35%, by weight based on total weight of the microemulsion.

(b)(iii) Hair Conditioning Agent

The hydrophilic phase of the water-in-oil microemulsion of the invention comprises a hair conditioning agent.

One suitable class of conditioning agent is a quaternary ammonium cationic surfactant.

Examples of suitable cationic surfactants of this type are those corresponding to the general formula:

[N(R₁)(R₂)(R₃)(R₄)]⁺(X)⁻

in which R₁, R₂, R₃, and R₄ are independently selected from (a) an aliphatic group of from 1 to 22 carbon atoms, or (b) an aromatic, alkoxy, polyoxyalkylene, alkylamido, hydroxyalkyl, aryl or alkylaryl group having up to 22 carbon atoms; and X is a salt-forming anion such as those selected from halogen, (e.g. chloride, bromide), acetate, citrate, lactate, glycolate, phosphate nitrate, sulphate, and alkylsulphate radicals.

The aliphatic groups can contain, in addition to carbon and hydrogen atoms, ether linkages, and other groups such as amino groups. The longer chain aliphatic groups, e.g., those of about 12 carbons, or higher, can be saturated or unsaturated.

Preferred cationic surfactants are monoalkyl quaternary ammonium compounds in which the alkyl chain length is C12 to C22.

Other preferred cationic surfactants are so-called dialkyl quaternary ammonium compounds in which R1 and R2 independently have an alkyl chain lengths from C12 to C22 and R3 and R4 have 2 or less carbon atoms.

Examples of suitable cationic surfactants include: cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, PEG-2 oleylammonium chloride and salts of these where the chloride is replaced by other halogen, (e.g. bromide), acetate, citrate, lactate, glycolate, phosphate nitrate, sulphate, or alkylsulphate. Other suitable cationic surfactants include those materials having the CTFA designations Quaternium-5, Quaternium-31 and Quaternium-18.

Preferred examples are cetyltrimethylammonium chloride, available commercially, for example as ARQUAD 16/29, from Akzo, and lauryl trimethylammonium chloride, available commercially, for example as ARQUAD C-35, from Akzo.

Another suitable class of conditioning agent is a cationic polymer.

By “cationic polymer” is meant any polymer containing cationic groups and/or groups that can be ionized into cationic groups.

Suitable cationic polymers may be homopolymers or may be formed from two or more types of monomers.

The weight average (M_{w} ) molecular weight of the cationic polymer is preferably between 300,000 and 2M Dalton, more preferably between 750,000 and 1.5M Dalton.

The cationic groups will generally be present as a substituent on a fraction of the total monomers of the cationic polymer. Thus when the polymer is not a homopolymer it can contain non-cationic spacer monomers. Such polymers are described in the CTFA Cosmetic Ingredient Dictionary, 3rd edition. The ratio of the cationic to non-cationic monomers is selected to give polymers having a cationic charge density in the required range.

The cationic charge density of the cationic polymer may suitably be determined via the Kjeldahl method as described in the US Pharmacopoeia under chemical tests for nitrogen determination. Preferred cationic polymers will have cationic charge densities of at least about 0.9 meq/gm, more preferably at least about 1.6 meq/gm, most preferably at least about 1.8 meq/g, but also preferably less than about 7 meq/gm, more preferably less than about 5 meq/gm, most preferably less than about 3.0 meq/g, as measured at the pH of intended use of the microemulsion. The pH of intended use of the microemulsion typically ranges from about pH 3 to about pH9, preferably from about pH4 to about pH7.

Any anionic counterions may be use in association with the cationic polymers so long as the cationic polymers remain soluble in the hydrophilic phase, and so long as the counterions are physically and chemically compatible with the essential components of the microemulsion or do not otherwise unduly impair product performance, stability or aesthetics. Examples of such counterions include: halides (e.g., chloride, fluoride, bromide, iodide), sulfate, methylsulfate, and mixtures thereof.

The preferred cationic polymers are chosen from those that contain units comprising primary, secondary, tertiary and/or quaternary amine groups that can either form part of the main polymer chain or can be borne by a side substituent directly connected thereto.

Suitable cationic polymers may be naturally-derived materials such as cationic polysaccharides.

Cationic polysaccharides suitable for use in compositions of the invention include monomers of the formula:

A-O—[R—N⁺(R¹)(R²)(R³)X⁻],

wherein: A is an anhydroglucose residual group, such as a starch or cellulose anhydroglucose residual. R is an alkylene, oxyalkylene, polyoxyalkylene, or hydroxyalkylene group, or combination thereof. R¹, R² and R³ independently represent alkyl, aryl, alkylaryl, arylalkyl, alkoxyalkyl, or alkoxyaryl groups, each group containing up to about 18 carbon atoms. The total number of carbon atoms for each cationic moiety (i.e., the sum of carbon atoms in R¹, R² and R³) is preferably about 20 or less, and X is an anionic counterion.

Preferred cationic polysaccharides are cationic cellulose derivatives such as those salts of hydroxyethyl cellulose reacted with trimethyl ammonium substituted epoxide, referred to in the industry (CTFA) as Polyquaternium 10. Specific examples of these materials include those polymers available from Amerchol Corporation in their Polymer JR series of polymers, such as Polymer JR125, Polymer JR400 and Polymer JR30M. Other suitable types of cationic cellulose include the polymeric quaternary ammonium salts of hydroxyethyl cellulose reacted with lauryl dimethyl ammonium-substituted epoxide referred to in the industry (CTFA) as Polyquaternium 24.

Another preferred class of cationic polysaccharide that can be used is a cationic guar gum derivative, especially guar hydroxypropyltrimethylammonium chloride. Specific examples of these materials include those polymers available from Rhodia in their JAGUAR series of polymers, such as JAGUAR C13S and JAGUAR C17.

Suitable cationic polymers may also be synthetically-derived materials such as those formed from vinyl monomers having cationic amine or quaternary ammonium functionalities, optionally together with non-cationic spacer monomers.

Suitable non-cationic spacer monomers include (meth)acrylamide, alkyl and dialkyl (meth)acrylamides, alkyl (meth)acrylate, vinyl caprolactone and vinyl pyrrolidine. The alkyl and dialkyl substituted monomers preferably have C1-C7 alkyl groups, more preferably C1-3 alkyl groups. Other suitable water soluble spacer monomers include vinyl esters, vinyl alcohol, maleic anhydride, propylene glycol and ethylene glycol.

Suitable vinyl monomers having cationic amine or quaternary ammonium functionalities include dialkylaminoalkyl acrylate, dialkylaminoalkyl methacrylate, dialkylaminoalkyl acrylamide, dialkylaminoalkyl methacrylamide, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts. The alkyl portions of these monomers are preferably lower alkyls such as the C1, C2 or C3 alkyls.

Examples of suitable cationic polymers formed from the above types of monomer include copolymers of 1-vinyl-2-pyrrolidone and 1-vinyl-3-methylimidazolium salt (e.g. chloride salt) (referred to in the industry by the Cosmetic, Toiletry, and Fragrance Association, “CTFA”, as Polyquaternium-16); copolymers of 1-vinyl-2-pyrrolidone and dimethylaminoethyl methacrylate (referred to in the industry by CTFA as Polyquaternium-11); cationic diallyl quaternary ammonium-containing polymers, including, for example, dimethyldiallylammonium chloride homopolymer, copolymers of acrylamide and dimethyldiallylammonium chloride (referred to in the industry by CTFA as Polyquaternium 6 and Polyquaternium 7, respectively); terpolymers of acrylic acid with dimethyldiallylammonium chloride and acrylamide (referred to in the industry by CTFA as Polyquaternium 39), and terpolymers of acrylic acid with methacrylamidopropyl trimethylammonium chloride and methyl acrylate (referred to in the industry by CTFA as Polyquaternium 47).

Mixtures of any of the above described hair conditioning agents may also be used.

The total amount of hair conditioning agent suitably ranges from 0.05 to 4%, preferably from 0.07 to 3%, by weight based on total weight of the microemulsion.

Process

Water-in-oil microemulsions according to the present invention form spontaneously and may be prepared by simple mixing at ambient temperature.

A preferred process for preparing a water-in-oil microemulsion according to the present invention comprises the following steps:

(I) forming a dispersion of the conditioning agent [(b)(iii)] in the water [(b)(i)];

(II) forming a separate mixture of the oil phase [(a)] and nonionic emulsifier [(b)(ii)];

(III) blending the dispersion obtained in (I) with the mixture obtained in (II).

Product Form and Usage

Compositions of this invention are preferably for application directly to the hair in neat form, either before or after shampooing.

Accordingly the invention also provides a method of treating hair comprising the step of applying a water-in-oil microemulsion as described above directly to the hair as a pre-wash treatment or as a post-wash treatment.

Optional Ingredients

Compositions of this invention may contain any other ingredient normally used in hair treatment formulations. These other ingredients may include preservatives such as phenoxetol® (2-phenoxyethanol), colouring agents, antioxidants such as BHT (butylhydroxytoluene), fragrances and antimicrobials such as Glycacil-L® (iodopropynyl butylcarbamate). Each of these ingredients will be present in an amount effective to accomplish its purpose. Generally these optional ingredients are included individually at a level of up to about 5% by weight based on total weight of the microemulsion.

The invention is further illustrated by way of the following Examples, in which all percentages are by weight based on total weight unless otherwise stated.

### EXAMPLES

Water-in-oil microemulsions containing various hair conditioning agents were prepared, having ingredients as shown in the following Table:

| Formulation | | | | |
|---|---|---|---|---|
| Examples: | | | | |
| Ingredient | Example 1 | Example 2 | Example 3 | Example 4 |
| Sunflower oil | 32.5 | 32.5 | 32.5 | 32.5 |
| Light mineral oil | 32.5 | 32.5 | 32.5 | 32.5 |
| (Sirius M40, from | | | | |
| Silkolene) | | | | |
| Nonionic emulsifier | 30.0 | 30.0 | 30.0 | 30.0 |
| (NEODOL 23-3, from | | | | |
| Shell Co.) | | | | |
| Water | to 100 | to 100 | to 100 | to 100 |
| Cationic surfactant | 2.5 (a.i.) | — | — | 2.5 (a.i.) |
| (ARQUAD C-35, from | | | | |
| Akzo) | | | | |
| Cationic surfactant | — | 2.5 (a.i.) | — | — |
| (ARQUAD 16/29, from | | | | |
| Akzo) | | | | |
| Cationic polymer | — | — | 0.075 | 0.075 |
| (JAGUAR C17, from | | | (a.i.) | (a.i.) |
| Rhodia) | | | | |

Comparative evaluations of the above formulations according to the invention were carried out using a control formulation of 50 wt % Sirius M40 and 50 wt % sunflower oil.

The formulations of Examples 1 to 4 were each compared against the control formulation across a number of performance attributes. Evaluation was carried out in two stages:

(i) Post Oiling.

Half of the hair of a mannequin head was oiled with the control formulation and the other half with the test formulation (Example 1, 2 or 3 respectively). 2.0 ml of formulation was used to oil the individual half head. After one hour the mannequin head was assessed by an expert salon hairdresser.

(ii) Post Wash.

3.5 ml of a commercial shampoo was measured and applied onto the oiled half head, followed by washing and rinsing in accordance with normal procedures. The shampooing and rinsing procedure was repeated for a second application. The same procedure was followed for the other oiled half head. After washing and rinsing was complete the mannequin head was allowed to dry at normal temperature (20 to 25 degrees C.). On drying the mannequin head was assessed by an expert salon hairdresser.

The following results were obtained:

Post Oiling:

Compared to the control, the formulation of Example 1 gave significantly (>90%) better hair body and significantly (>90%) reduced hair sticky feel. The formulation of Example 1 was also found to have significantly (>90%) reduced product sticky feel compared to the control.

Compared to the control, the formulation of Example 2 gave significantly (>99%) better hair body. The formulation of Example 2 was also found to have significantly (>90%) reduced product sticky feel compared to the control.

Compared to the control, the formulation of Example 3 gave significantly (>99%) better hair body and significantly (>90%) reduced hair sticky feel.

Compared to the control, the formulation of Example 4 gave significantly (>95%) better hair conditioning and significantly (>90%) better hair shine.

Post Wash:

Compared to the control, the formulation of Example 1 gave significantly (>95%) better hair body, significantly (>90%) better hair conditioning and significantly (>95%) better hair shine.

Compared to the control, the formulation of Example 2 gave significantly (>90%) better hair body, significantly (>90%) better hair conditioning and significantly (>95%) better hair shine.

Compared to the control, the formulation of Example 3 gave significantly (>95%) better hair body and significantly (>90%) better hair shine.

Compared to the control, the formulation of Example 4 gave significantly (>99%) better hair body, significantly (>90%) better hair conditioning and significantly (>90%) better hair shine.

1. A water-in-oil microemulsion for hair treatment comprising:
(a) an oil phase comprising:
(i) a first oily component which is one or more glyceride fatty esters, and
(ii) a second oily component which is one or more hydrocarbon oils of average carbon chain length less than 20 carbon atoms, and
(b) a hydrophilic phase comprising:
(i) water,
(ii) a nonionic emulsifier which is an ethoxylated alcohol having an HLB of at least 6, and
(iii) a hair conditioning agent.

2. A microemulsion according to claim 1, in which the source of glyceride fatty esters is selected from coconut oil, sunflower oil, almond oil and mixtures thereof.

3. A microemulsion according to claim 1, in which the total content of glyceride fatty ester ranges from 20% to 80% by weight based on total weight of the microemulsion.

4. A microemulsion according to claim 1, in which the hydrocarbon oil is light mineral oil.

5. A microemulsion according to claim 1, in which the total content of hydrocarbon oil ranges from 20% to 80% by weight based on total weight of the microemulsion.

6. A microemulsion according to claim 1, in which the glyceride fatty ester:hydrocarbon oil weight ratio ranges from 95:5 to 5:95, preferably from 90:10 to 10:90, most preferably from 80:20 to 20:80.

7. A microemulsion according to claim 1, in which the water level ranges from 3 to 7%, more preferably from 4 to 6% by weight based on total weight of the microemulsion.

8. A microemulsion according to claim 1, in which the HLB value of the ethoxylated alcohol ranges from 6 to 12, preferably from 7 to 10, more preferably from 7 to 9.

9. A microemulsion according to claim 8, in which the ethoxylated alcohol is a higher aliphatic, primary alcohol containing about 9 to 15 carbon atoms, condensed with about 2.5 to 10 moles of ethylene oxide.

10. A microemulsion according to claim 9, in which the ethoxylated alcohol is C12 to 13 alkanol condensed with 3 moles ethylene oxide.

11. A microemulsion according to claim 1, in which the hair conditioning agent is a quaternary ammonium cationic surfactant.

12. A microemulsion according to claim 11, in which the quaternary ammonium cationic surfactant is a monoalkyl quaternary ammonium compound in which the alkyl chain length is C12 to C22.

13. A microemulsion according to claim 1, in which the hair conditioning agent is a cationic polymer.

14. A microemulsion according to claim 13, in which the cationic polymer is a cationic guar gum derivative, especially guar hydroxypropyltrimethylammonium chloride.

15. A method of treating hair comprising the step of applying a water-in-oil microemulsion according to claim 1 directly to the hair as a pre-wash treatment or as a post-wash treatment.
